(19) United States
(12) Patent Application Publication
(10) Pub. No.: US 2008/0154075 A1
(43) Pub. Date: Jun. 26, 2008

(54) Process for the Production of Olefins

(76) Inventors: Bryce Alan Williams, Lisle, IL (US); Ian Raymond Little, Naperville, IL (US)

Correspondence Address: NIXON & VANDERHYE, PC, 901 NORTH GLEBE ROAD, 11TH FLOOR, ARLINGTON, VA, 22203, US

(21) Appl. No.: 11/921,319

(22) PCT Filed: Jun. 28, 2006
(86) PCT No.: PCT/GB2006/002371

§ 371 Date: Dec. 20, 2007

Related U.S. Application Data
(60) Provisional application No. 60/696,851, filed on Jul. 6, 2005.

(51) Int. Cl. C07C 4/06

(52) U.S. Cl. 585/302

(57) ABSTRACT

The present invention provides a process for the production of olefins, which process comprises: (a) providing an autothermal cracking unit having at least two reactors, (b) autothermally cracking a first hydrocarbon stream by contacting said stream with a first catalyst bed in the presence of molecular oxygen containing gas in one or more first reactors to produce one or more first product streams, (c) autothermally cracking a second hydrocarbon stream by contacting said stream with a second catalyst bed in the presence of molecular oxygen containing gas in one or more second reactors to produce one or more second product streams, (d) separating at least one olefin-containing product stream from the first and second product streams, characterised in that at least two of the following apply: (i) the first and second catalyst beds are different, (ii) the first and second hydrocarbon streams are different, and (iii) the second hydrocarbon stream is not cracked to optimize production of C2 to C4 olefins.

The present invention relates to a process for the production of olefins, and, in particular, to a process for the production of olefins by autothermal cracking.

Autothermal cracking is a route to olefins in which a hydrocarbon feed is mixed with oxygen and passed over an autothermal cracking catalyst. The autothermal cracking catalyst is capable of supporting combustion beyond the fuel rich limit of flammability. In autothermal cracking, combustion is initiated on the catalyst surface and the heat required to raise the reactants to the process temperature and to carry out the endothermic cracking process is generated in situ. The autothermal cracking of paraffinic hydrocarbons is described, for example, in EP-332289B; EP-529793B; EP-709446A and WO 00/14035.

Autothermal cracking may be used to crack gaseous paraffinic hydrocarbons or mixtures thereof, such as ethane, propane and butane, to produce ethylene, propene and butene, or to crack liquid paraffinic hydrocarbons or mixtures thereof, such as naphthas, gas oils and FT liquids.

It has now been found that the autothermal cracking process may be advantageously operated by cracking hydrocarbon feeds using different catalyst beds and/or to produce different product streams.

Thus, the present invention provides a process for the production of olefins, which process comprises:

(a) providing an autothermal cracking unit having at least two reactors,
(b) autothermally cracking a first hydrocarbon stream by contacting said stream with a first catalyst bed in the presence of molecular oxygen containing gas in one or more first reactors to produce one or more first product streams,
(c) autothermally cracking a second hydrocarbon stream by contacting said stream with a second catalyst bed in the presence of molecular oxygen containing gas in one or more second reactors to produce one or more second product streams,
(d) separating at least one olefin-containing product stream from the first and second product streams,
characterised in that at least two of the following apply:

(i) the first and second catalyst beds are different,

(ii) the first and second hydrocarbon streams are different, and

(iii) the second hydrocarbon stream is not cracked to optimise production of C2 to C4 olefins.

“Autothermally cracking” as used herein refers to cracking in which combustion of the reactant feed is initiated on the catalyst surface and the heat required to raise the reactants to the process temperature and to carry out the endothermic cracking to produce olefins is generated in situ.

By “not cracked to optimise production of C2 to C4 olefins” is meant that the cracking of the second hydrocarbon stream is not optimised for production of any of the individual C2 to C4 olefins, such as ethylene, or combinations thereof, such as total C2 to C4 olefins. Thus, the second hydrocarbon stream is cracked to produce one or more second product streams using a second catalyst bed and/or under conditions which are selected to produce a product other than a C2 to C4 olefin. C2 to C4 olefins may still be produced in addition to the product other than a C2 to C4 olefin, but the catalyst bed and/or conditions are selected to enhance production of a product other than a C2 to C4 olefin relative to the optimum catalyst bed and conditions for production of said C2 to C4 olefin.

The autothermal cracking unit comprises at least two reactors. Typically two to eight reactors would be present on a commercial cracking unit.

For avoidance of doubt, the one or more first reactors and the one or more second reactors in present invention operate in parallel to crack the respective first and second hydrocarbon streams.

The present invention allows an autothermal cracking unit comprising at least two reactors to operate with a high degree of flexibility in both the choice of hydrocarbon feedstocks and in the selection of products to be produced to maximise the overall value of the cracking process.

In a first aspect, the present invention provides a process for the production of olefins, which process comprises:

(a) providing an autothermal cracking unit having at least two reactors,
(b) autothermally cracking a first hydrocarbon stream by contacting said stream with a first catalyst bed in the presence of molecular oxygen containing gas in one or more first reactors to produce one or more first product streams,
(c) autothermally cracking a second hydrocarbon stream by contacting said stream with a second catalyst bed in the presence of molecular oxygen containing gas in one or more second reactors to produce one or more second product streams,
(d) separating at least one olefin-containing product stream from the first and second product streams,
characterised in that the first and second hydrocarbon streams are different, that the second hydrocarbon stream is not cracked to optimise production of C2 to C4 olefins and that an olefin-containing product stream comprising a product which is not a C2 to C4 olefin is separated in step (d).

In this aspect, different first and second hydrocarbon streams are fed to the respective reactors (sets of reactors) and at least the second hydrocarbon stream is not cracked to optimise production of C2 to C4 olefins.

For example, the second hydrocarbon stream may be cracked to enhance the yield of one or more products selected from aromatic compounds, dienes, acetylenes, and synthesis gas relative to the optimum ethylene, propene and/or butene yield.

Preferably, the second hydrocarbon stream is cracked to produce one or more second product streams comprising one of more linear alpha-olefins having at least 6 carbon atoms.

In a first preferred example, the second hydrocarbon stream comprises one or more linear alpha-olefins (LAO's) and is cracked to produce linear alpha olefins having a lower number of carbon atoms than the LAO's in the second hydrocarbon stream.

In this example, the LAO's in the second hydrocarbon stream may comprise the “higher” LAO's formed from ethylene oligomerisation. The LAO products from a conventional ethylene oligomerisation process generally comprise a distribution of LAO's of general formula C₂ₙH₍₄ₙ₊₁₎CH═CH₂, where n is 1, 2, 3, etc., i.e. 1-butene, 1-hexene, 1-octene etc.

LAO's may be used as surfactants and lubricants, but the most valuable uses of the “lower” LAO's, especially 1-hexene and 1-octene are as co-monomers for polymer production. The “higher” LAO's are generally less valuable.

(“Lower” LAO and “higher” LAO as used herein refer to the relative number of carbon atoms in the respective LAO's.)

It is, however, difficult to target specific LAO's by ethylene oligomerisation due to the inherent product distribution obtained, and hence, significant proportions of higher LAO's are obtained. A typical product distribution may contain approximately 70% C10 and lower LAO's, 20% C12 and C14, 5% C16 and C18 and 5% C20 and above. In the process of the present invention, “higher” LAO's may be cracked to produce the more valuable “lower” LAO's in the one or more second reactors. Suitably, the second hydrocarbon stream comprises LAO's having at least 10 carbon atoms, such as 1-decene and above, and is cracked to produce 1-octene and/or 1-hexene.

In a second preferred example, the second hydrocarbon stream comprises linear paraffinic hydrocarbons and is cracked to produce linear alpha olefins in the one or more second reactors. The production of linear alpha-olefins (LAO's) and ethylene by autothermal cracking of paraffinic hydrocarbons, has been described, for example, in US2004/0199038.

Most preferably, the linear paraffinic hydrocarbons may be derived from the product stream of a Fischer-Tropsch process. The Fischer-Tropsch process produces hydrocarbons from carbon monoxide and hydrogen (synthesis gas). Typically the product stream from a Fischer-Tropsch reactor includes C4 to C20+ hydrocarbons. The hydrocarbons are generally highly linear. Depending on the catalyst and process used, the product stream may be highly paraffinic or may comprise a substantial proportion of olefins.

The full range FT product stream (i.e. C4 to C20+ hydrocarbons) may be used as the second hydrocarbon stream or the FT product stream may be treated by any suitable technique, for example by distillation, to remove components thereof prior to use as the second hydrocarbon stream. One advantage of this embodiment is that the FT-derived feed need not be treated to reduce the olefin content, for example by hydrotreatment or hydrocracking, prior to being fed to the autothermal cracking process. In contrast, it is conventional to treat FT-derived streams to reduce the olefins content therein prior to steam cracking because of the propensity of the olefins to cause coking in the steam cracker.

As one example, the FT product stream may be treated by distillation to remove the C8 and lower components and produce a fraction comprising the C9 and heavier components which may be used, without treatment to reduce the olefin content thereof, as the second hydrocarbon stream. This second hydrocarbon stream is autothermally cracked to produce a second product stream comprising 1-octene and 1-hexene.

By “the first and second hydrocarbon streams are different” is meant that the compositions of the second hydrocarbon stream and the first hydrocarbon stream differ. The difference in composition may be in the presence of one or more components in either the first or second hydrocarbon stream that are absent in the other stream and/or in the relative amounts of components that are present in both streams.

In general, the first hydrocarbon stream will be “lighter” than the second hydrocarbon stream in this first aspect. “Lighter” (and “heavier”) as used herein, relate to the relative average carbon number of the components in the first and second hydrocarbon streams. Thus, the first hydrocarbon stream being “lighter” than the second hydrocarbon stream means that the average carbon number of the components in the first hydrocarbon stream is lower than the average carbon number of the components in the second hydrocarbon stream.

For example, both the first hydrocarbon stream and second hydrocarbon stream may comprise liquid hydrocarbons but the first hydrocarbon stream will comprise “lighter” liquid hydrocarbons than the second hydrocarbon stream. As one example of this option, both the first and second hydrocarbon streams may be derived from an FT product stream which is treated by distillation to remove the C8 and lower components and produce a fraction comprising the C9 and heavier components which may be used as the second hydrocarbon stream, as described above, and wherein the removed C8 and lower fraction of the FT product stream is used as the first hydrocarbon stream.

Preferably, the second hydrocarbon stream comprises liquid paraffinic hydrocarbons and the first hydrocarbon stream comprises gaseous paraffinic hydrocarbons.

Preferably, the first hydrocarbon stream is cracked in the one or more first reactors using a first catalyst and under conditions selected to produce one or more olefins selected from C2 to C4 olefins.

The first and second catalyst beds in this embodiment may be the same or may be different. By “the same” is meant to comprise the same active metal or metals, and, where present, any promoters and supports, and in essentially the same relative amounts. Otherwise the catalyst beds are considered to be different.

Although the selection of catalyst bed can also influence the products obtained, the conditions used in each set of reactors will generally be significantly different. The conditions generally, will include the hydrocarbon to oxygen ratio, hydrogen (where present) to oxygen ratio, reaction pressure and space velocity/contact time.

Thus, the first and second catalyst beds may be the same, and variation in the product streams will be provided by the use of different first and second hydrocarbon streams, and different reaction conditions in the first and second reactors to produce the desired respective product streams.

Alternatively, the first and second catalyst beds may be different. The selection of suitable catalyst beds in this case may be determined by the feedstream to be treated and/or by the products it is desired to produce.

Thus, where the first hydrocarbon stream and the second hydrocarbon stream are different the respective catalyst beds may be selected in view of the compositions of the respective feeds. As one example, where the first hydrocarbon stream comprises carbon monoxide, which can potentially poison certain autothermal cracking catalysts, but the second hydrocarbon stream does not, the first catalyst bed may comprise a composition, such as a mixture of platinum and palladium, which has been found to be tolerant to carbon monoxide, but the second catalyst bed can be selected without this constraint.

As an alternative example, where the first hydrocarbon stream comprises gaseous paraffinic hydrocarbons and the second hydrocarbon stream comprises liquid paraffinic hydrocarbons, the second catalyst bed may comprise a composition which is selected for cracking of liquid feeds, for example, has reduced coking tendencies, but the first catalyst bed can be selected without this constraint.

In contrast, if the respective first and second hydrocarbon streams were mixed prior to cracking and the mixed stream passed to all reactors, not only would cracking have to be performed under sub-optimal conditions compared to the separate cracking of the respective first and second hydrocarbon streams, but the choice of catalyst bed would be constrained for all reactors by components that may only be present in one of the respective feedstreams.

Alternatively, or in addition, the first and second catalyst beds may be selected based on their selectivity for the desired products from the respective feedstreams. In an example of this embodiment, the first hydrocarbon stream is cracked in the one or more first reactors using a first catalyst bed and under conditions selected to produce one or more olefins selected from C2 to C4 olefins and the second hydrocarbon stream is cracked in the one or more second reactors to produce one or more second product streams using a second catalyst bed and under conditions which are selected to produce a product other than a C2 to C4 olefin.

As an example of this embodiment, the second catalyst bed may be selected based on suitability for the production of linear alpha-olefins having at least 6 carbon atoms as described above (i.e. from either higher LAO's or from an FT-derived feedstream).

Thus, different products may be advantageously produced from the different reactors by selection of catalyst bed. In contrast, although it might be possible to produce mixed product streams from the reactors by operating them all to produce the same product stream, neither product would be being produced under optimal conditions compared to the separate cracking of the present invention.

In a second aspect, the present invention provides a process for the production of olefins, which process comprises:

(a) providing an autothermal cracking unit having at least two reactors,
(b) autothermally cracking a first hydrocarbon stream by contacting said stream with a first catalyst bed in the presence of molecular oxygen containing gas in one or more first reactors to produce one or more first product streams,
(c) autothermally cracking a second hydrocarbon stream by contacting said stream with a second catalyst bed in the presence of molecular oxygen containing gas in one or more second reactors to produce one or more second product streams,
(d) separating at least one olefin-containing product stream from the first and second product streams,
characterised in that the first and second catalyst beds are different and that either
(i) the first and second hydrocarbon streams are different, or
(ii) the second hydrocarbon stream is not cracked to optimise production of C2 to C4 olefins.

In this aspect, different first and second product streams will be produced.

The selection of suitable catalyst beds in this case may be determined by the feedstream to be treated and/or by the products it is desired to produce, as described above.

Preferably the first and second catalyst beds differ in at least one of the following properties:

(a) different active metals,
(b) different promoters (or absence/presence of a promoter),
(c) different supports (or absence/presence of a support).

Different supports, as used herein, may refer to supports with different chemical or with different physical properties, such as support structure, geometry or pore size.

In one embodiment of this second aspect, the first and second hydrocarbon streams may be the same but will be cracked using different catalyst beds and under different conditions to produce different product streams, wherein at least the second hydrocarbon stream is not cracked to optimise production of C2 to C4 olefins.

In this embodiment, the first hydrocarbon stream is preferably cracked in the one or more first reactors using a first catalyst bed and under conditions selected to produce one or more olefins selected from C2 to C4 olefins.

As an example, where the first hydrocarbon stream and second hydrocarbon stream are the same and comprise liquid hydrocarbons, the first hydrocarbon stream may be cracked in the one or more first reactors using a first catalyst bed and under conditions selected to produce one or more olefins selected from C2 to C4 olefins, and the second hydrocarbon stream may be cracked to produce one or more second product streams using a second catalyst bed and under conditions which are selected to produce a product other than a C2 to C4 olefin.

In this embodiment, the second hydrocarbon stream may be cracked to enhance the yield of one or more products selected from aromatic compounds, dienes, acetylenes, and synthesis gas relative to the optimum ethylene, propene and/or butene yield. More preferably, the second hydrocarbon stream is cracked to produce one or more second product streams comprising one of more linear alpha-olefins having at least 6 carbon atoms, and most preferably, by cracking of either one or more linear alpha-olefins (LAO's) to produce linear alpha olefins having a lower number of carbon atoms than the LAO's in the second hydrocarbon stream or linear paraffinic hydrocarbons to produce linear alpha-olefins, as described above.

In an alternative embodiment, the first and second hydrocarbon streams may be different, and will be cracked using different catalyst beds under different conditions to produce different product streams. For example, the first hydrocarbon stream may be a gaseous hydrocarbon and the second hydrocarbon stream may be a liquid hydrocarbon. In this embodiment, both the first and second hydrocarbon streams may be cracked to produce first and second product streams comprising one or more C2 to C4 olefins.

In step (d) of the process of the present invention, at least one olefin-containing stream is separated from the first and second product streams.

This may be achieved by separately treating the first and second product streams to separate the olefins in each stream from any by-products or unreacted components as separate first and second olefin-containing product streams. Alternatively, it may be desirable that the first and second product streams are combined and treated to produce the at least one olefin-containing product stream. For example, where the first hydrocarbon stream comprises gaseous paraffinic hydrocarbons and is cracked in the one or more first reactors using a first catalyst bed and under conditions selected to produce one or more olefins selected from C2 to C4 olefins and the second hydrocarbon stream comprises liquid paraffinic hydrocarbons and is cracked in the one or more second reactors using a second catalyst bed and under conditions selected to produce a product other than a C2 to C4 olefin, the first and second product streams may be combined and treated to produce a first olefin-containing product stream comprising one or more C2 to C4 olefins and a second olefin-containing product stream comprising a product which is other than a C2 to C4 olefin. This has the advantage that the separate hydrocarbon feedstreams may be cracked to produce separate product streams, but that said streams may be treated and the required components separated in the same separations train, avoiding duplication of separations equipment.

As noted previously, the first and second catalyst beds and/or the conditions in the first and second (sets of) reactors in steps (b) and (c) may be selected based on the first and second feedstreams and/or the first and second product streams.

In general, the oxygen-containing gas in each set of reactors may be provided as any suitable molecular oxygen containing gas, such as molecular oxygen itself or air.

Preferably, hydrogen is co-fed to each set of reactors. Hydrogen co-feeds are advantageous because, in the presence of the catalysts, the hydrogen combusts preferentially relative to hydrocarbons in the respective feedstreams, thereby increasing the selectivity of the overall process. The amount of hydrogen combusted may be used to control the amount of heat generated and hence the severity of cracking, which may be used to favour different products.

Thus, the molar ratios of hydrogen to oxygen can vary over any operable ranges provided that the desired product streams are produced. Suitably, the molar ratios of hydrogen to oxygen in each set of reactors are in the range 0.2 to 4, preferably, in the range 0.2 to 3.

The hydrocarbons to be cracked (first and second hydrocarbon streams) and molecular oxygen-containing gas may be contacted with the respective first and second catalyst beds in any suitable molar ratios, provided that the product streams comprising the desired products are produced. The preferred stoichiometric ratios of hydrocarbon to oxygen-containing gas in each set of reactors are generally 5 to 16, preferably 5 to 13.5 times, and more preferably 6 to 10 times, the stoichiometric ratio of hydrocarbon to oxygen-containing gas required for complete combustion of the hydrocarbon to carbon dioxide and water.

The hydrocarbons to be cracked (first and second hydrocarbon streams), the molecular oxygen-containing gas and any other feed components are preferably pre-heated prior to contact with the catalyst. Pre-heating may be performed before or after mixing of the reactants.

The hydrocarbon streams are each passed over the respective catalyst beds at gas hourly space velocities of greater than 10,000 h⁻¹, preferably above 20,000 h⁻¹ and most preferably, greater than 100,000 h⁻¹. It will be understood, however, that the optimum gas hourly space velocities will depend upon the respective pressures.

The autothermal cracking steps are usually operated at a pressure of greater than 0.5 barg. Preferably the autothermal cracking steps are operated at a pressure of between 0.5-40 barg.

In general, as would be apparent to the person skilled in the art, the most preferred conditions will be dependent on the product stream it is desired to produce. For example, production of LAO's having at least 6 carbon atoms generally requires less severe conditions than production of ethylene. Thus, production of LAO's will preferably utilise hydrogen to oxygen ratios at the lower end of the preferred ranges and hydrocarbon to oxygen ratios at the higher end of the preferred ranges for these variables.

The severity of reaction will be reflected in the catalyst bed exit temperature and the catalyst bed exit temperature may be correspondingly lower for LAO production than for ethylene, for example.

The actual catalyst bed exit temperature may depend on a number of factors other than reaction severity, such as heat losses in the reactor, but in general, the autothermal cracking steps may suitably be carried out at catalyst bed exit temperatures in the range 600° C. to 1200° C.

For production of C2 to C4 olefins the catalyst bed exit temperatures are preferably in the range 800° C. to 1050° C. and, most preferably, in the range 800° C. to 1000° C. In contrast, for production of LAO's having at least 6 carbon atoms the catalyst bed exit temperatures are preferably in the range 600° C. to 1000° C. and, most preferably, in the range 650° C. to 900° C.

For production of synthesis gas, relatively severe conditions are favoured and the catalyst bed exit temperatures are preferably in the range 900° C. to 1200° C. and, most preferably, in the range 100° C. to 1150° C.

The first and second catalyst beds are each capable of supporting combustion beyond the normal fuel rich limit of flammability.

The catalyst beds may, independently, comprise a single bed of catalyst, or may comprise sequential beds of catalysts, as described, for example, in WO 02/04389.

Particularly suitable catalysts comprise Group VIII metals as their catalytic components (referred to as “Group VIII catalysts”). Suitable Group VIII metals include platinum, palladium, ruthenium, rhodium, osmium and iridium. Rhodium, and more particularly, platinum and palladium are preferred. Typical Group VIII metal loadings range from 0.01 to 100 wt %, preferably, between 0.01 to 20 wt %, and more preferably, from 0.01 to 10 wt % based on the total dry weight of the catalysts.

Where a Group VIII catalyst is employed, it is preferably employed in combination with a catalyst promoter. The promoter may be a Group IIIA, IVA, and/or VA metal. Alternatively, the promoter may be a transition metal; the transition metal promoter being a different metal to that which may be employed as the Group VIII transition metal catalytic component. Preferred promoters are selected from the group consisting of Ga, In, Sn, Ge, Ag, Au or Cu. The atomic ratio of Group VIII B metal to the catalyst promoter may be 1:0.1-50.0, preferably, 1:0.1-12.0.

Preferred examples of promoted catalysts include Pt/Ga, Pt/In, Pt/Sn, Pt/Ge, Pt/Cu, Pd/Sn, Pd/Ge, Pd/Cu, Rh/Sn, Pt/Pd/Cu and Pt/Pd/Sn catalysts.

For the avoidance of doubt, the Group VIII metal and promoter in the catalysts may be present in any form, for example, as metals, or in the form of a metal compounds, such as oxides.

The catalysts may be unsupported, such as in the form of metal gauzes, but are preferably supported. Any suitable supports may be used such as ceramic or metal supports, but ceramic supports are generally preferred. Where ceramic supports are used, the composition of the ceramic supports may be any oxide or combination of oxides that is stable at high temperatures of, for example, between 600° C. and 1200° C. The support materials preferably have a low thermal expansion co-efficient, and are resistant to phase separation at high temperatures.

Suitable ceramic supports include corderite, lithium aluminium silicate (LAS), alumina (α-Al₂O₃), yttria stabilised zirconia, alumina titanate, niascon, and calcium zirconyl phosphate. The ceramic supports may be wash-coated, for example, with γ-Al₂O₃.

The catalysts may be prepared by any method(s) known in the art. For example, gel methods and wet-impregnation techniques may be employed. Typically, supports are impregnated with one or more solutions comprising the required metals, dried and then calcined in air. The supports may be impregnated in one or more steps. Preferably, multiple impregnation steps are employed. The supports are preferably dried and calcined between each impregnation, and then subjected to a final calcination, preferably, in air. The calcined supports may then be reduced, for example, by heat treatment in a hydrogen atmosphere.

The first and second product streams are quenched as they emerge from the respective reactors to avoid further reactions taking place. Usually the product streams are cooled to between 750-600° C. within less than 100 milliseconds of formation, preferably within 50 milliseconds of formation and most preferably within 20 milliseconds of formation e.g. within 10 milliseconds of formation. The heat from the quenching may be used to generate high-pressure steam, which can be used to provide power for those parts of the overall process requiring it.

Typically, each autothermal cracking reactor will comprise a product quench section immediately downstream of the catalyst. Each reactor will usually also comprise a feed mixing and pre-heat section or sections upstream of the catalyst.

Alternatively, it may be possible in certain circumstances to have common mixing, preheat and/or quenching sections for more than one reactor.

The first and second product streams may comprise, in addition of olefins, hydrogen, carbon monoxide, methane, and small amounts of acetylenes, aromatics and carbon dioxide.

In step (d) at least one olefin-containing product stream is separated from the first and second product streams.

This may be achieved by any suitable technique or series of techniques (on each separate stream or on the combined stream, as suitable). For example, an amine wash may be used to remove carbon dioxide and water, a demethaniser to remove hydrogen, carbon monoxide and methane, and hydrogenation to remove acetylenic compounds and dienes.

Any suitable treatments to separate the respective olefinic products may be used depending on the respective products themselves, but generally, for example, any ethylene will be separated using a deethaniser, and any propylene using a depropaniser.

The autothermal cracking of the second hydrocarbon stream will also result in a general lowering of the average carbon number of the remaining components of the second hydrocarbon stream. Thus, where the second hydrocarbon stream is heavier than the first hydrocarbon stream, the autothermal cracking of the second hydrocarbon stream will result in a general lowering of the average carbon number of the remaining components of the second hydrocarbon stream and make these components more suitable for cracking in the one or more first reactors. Hence, the one or more second reactors may also effectively act to treat the second hydrocarbon stream to make components of it more suitable for cracking in the one or more first reactors. In a preferred embodiment of the process of the present invention these components are recycled as a recycle stream to the one or more first reactors.

Thus, in a third aspect, the present invention provides a process for the production of olefins, which process comprises:

(a) providing an autothermal cracking unit having at least two reactors,
(b) autothermally cracking a first hydrocarbon stream by contacting said stream with a first catalyst in the presence of molecular oxygen containing gas in one or more first reactors to produce one or more first product streams using a first catalyst and/or under conditions which are selected to produce one or more olefins selected from C2 to C4 olefins,
(c) autothermally cracking a second hydrocarbon stream, which is heavier than the first hydrocarbon stream, by contacting said stream with a second catalyst in the presence of molecular oxygen containing gas in one or more second reactors to produce one or more second product streams using a second catalyst and/or under conditions which are selected to produce a product other than a C2 to C4 olefin,
(d) separating from the first and second product streams at least a first olefin-containing product stream comprising one or more olefins selected from C2 to C4 olefins, a second olefin-containing product stream comprising a product which is other than a C2 to C4 olefin and a recycle stream comprising components lighter than the second hydrocarbon stream, and
(e) recycling the recycle stream to one or more of the one or more first reactors.

In step (d) of this aspect of the present invention, a recycle stream is produced which is recycled to the one or more first reactors. Said stream will generally comprise components of the second product stream, other than the desired products, which have lower carbon number than the components of the second hydrocarbon stream i.e. which have been cracked, but not to the desired products.

Step (d) may comprise separately treating the first and second product streams to separate the respective streams or it may be desirable that the first and second product streams are combined and treated to separate the respective streams as previously described.

A second recycle stream comprising heavier components of the second product stream, for example unreacted components of the second hydrocarbon stream, may also be separated and recycled to the one or more second reactors.

The preferred embodiments of the first and second hydrocarbon streams, first and second catalysts and first and second product streams of steps (b) and (c) in this aspect are as described above.

Thus, preferably, the second hydrocarbon stream comprises liquid paraffinic hydrocarbons and is cracked to deliberately produce one or more products selected from aromatic compounds, dienes, acetylenes, synthesis gas and linear alpha olefins (comprising LAO's having at least 6 carbon atoms).

1-12. (canceled)

13. A process for the production of olefins, which process comprises:
(a) providing a single autothermal cracking unit having at least two reactors,
(b) autothermally cracking a first hydrocarbon stream by contacting said stream with a first catalyst in the presence of molecular oxygen containing gas in one or more first reactors of said autothermal cracking unit to produce one or more first product streams,
(c) autothermally cracking a second hydrocarbon stream by contacting said stream with a second catalyst in the presence of molecular oxygen containing gas in one or more second reactors of said autothermal cracking unit to produce one or more second product streams, said one or more second reactors operating in parallel to the one or more first reactors to crack the respective hydrocarbon streams, and
(d) separating at least one olefin-containing product stream from the first and second product streams, characterised in that at least two of the following apply:
(i) the first and second catalysts are different,
(ii) the first and second hydrocarbon streams are different, and
(iii) the second hydrocarbon stream is not cracked to optimise production of C2 to C4 olefins.

14. A process according to claim 13, wherein the first and second hydrocarbon streams are different, the second hydrocarbon stream is not cracked to optimise production of C2 to C4 olefins and an olefin-containing product stream comprising a product which is not a C2 to C4 olefin is separated in step (d).

15. A process as claimed in claim 13 wherein, the first hydrocarbon stream comprises lighter hydrocarbons than the second hydrocarbon stream.

16. A process as claimed in claim 13 wherein, the first hydrocarbon stream is cracked in the one or more first reactors using a first catalyst and under conditions selected to produce one or more olefins selected from C2 to C4 olefins.

17. A process according to claim 16, wherein the second hydrocarbon stream is heavier than the first hydrocarbon stream, and which process comprises the steps of:
(a) providing a single autothermal cracking unit having at least two reactors,
(b) autothermally cracking a first hydrocarbon stream by contacting said stream with a first catalyst in the presence of molecular oxygen containing gas in one or more first reactors of said autothermal cracking unit to produce one or more first product streams using a first catalyst and/or under conditions which are selected to produce one or more olefins selected from C2 to C4 olefins,
(c) autothermally cracking a second hydrocarbon stream, which is heavier than the first hydrocarbon stream, by contacting said stream with a second catalyst in the presence of molecular oxygen containing gas in one or more second reactors of said autothermal cracking unit to produce one or more second product streams using a second catalyst and/or under conditions which are selected to produce a product other than a C2 to C4 olefin,
(d) separating from the first and second product streams at least a first olefin-containing product stream comprising one or more olefins selected from C2 to C4 olefins, a second olefin-containing product stream comprising a product which is other than a C2 to C4 olefin, and a recycle stream comprising components lighter than the second hydrocarbon stream, and
(e) recycling the recycle stream to one or more of the one or more first reactors.

18. A process according to claim 13, wherein the first and second catalysts are different and either:
(i) the first and second hydrocarbon streams are different, or
(ii) the second hydrocarbon stream is not cracked to optimise production of C2 to C4 olefins.

19. A process as claimed in claim 13 wherein, the second hydrocarbon stream comprises liquid hydrocarbons.

20. A process as claimed in claim 19, wherein the second hydrocarbon stream is cracked to produce one or more second product streams comprising one of more linear alpha-olefins having at least 6 carbon atoms.

21. A process as claimed in claim 20, wherein the second hydrocarbon stream comprises one or more linear alpha-olefins (LAO's) and is cracked to produce linear alpha olefins having a lower number of carbon atoms than the LAO's in the second hydrocarbon stream.

22. A process as claimed in claim 20 wherein the second hydrocarbon stream comprises linear paraffinic hydrocarbons and is cracked to produce linear alpha olefins in the one or more second reactors.

23. A process as claimed in claim 22 wherein the linear paraffinic hydrocarbons are derived from the product stream of a Fischer-Tropsch process.

24. A process as claimed in claim 13 wherein, the first hydrocarbon stream is a gaseous hydrocarbon.
